(19) United States
(12) Patent Application Publication
(10) Pub. No.: US 2026/0083433 A1
(43) Pub. Date: Mar. 26, 2026

(54) ULTRASOUND PROBE WITH IMPROVED CENTER POSITION DETECTION

(71) Applicant: GE Precision Healthcare LLC, Waukesha, WI (US)

(72) Inventors: Christian Heinrich, Neukirchen (AT); Krystof Jezek, Vocklabruck (AT); Bernhard Aschenbrenner, Altmuenster am Traunsee (AT); Reinhard Wimmer, Aurolzmuenster (AT); Wolfgang Knoll, Zipf (AT)

(73) Assignee: GE Precision Healthcare LLC, Waukesha, WI (US)

(21) Appl. No.: 19/335,831

(22) Filed: Sep. 22, 2025

Related U.S. Application Data
(60) Provisional application No. 63/698,016, filed on Sep. 23, 2024.

(51) Int. Cl. A61B 8/00; A61B 8/12

(52) U.S. Cl. CPC A61B 8/4254; A61B 8/12; A61B 8/4444; A61B 8/461

(57) ABSTRACT

Systems and methods for providing an ultrasound imaging system with improved center position detection are described herein. In one example, the ultrasound imaging system includes a probe with a distal portion, a proximal portion, and a processing circuit. The distal portion includes a first magnet and a first sensor. The proximal portion is positioned at a head of the probe and includes a transducer, a second magnet, and a second sensor. The processing circuit is configured to detect a first voltage signal edge via the first magnet and the first sensor, rotate the transducer from a first position to a second position to locate a second voltage signal edge via the second magnet and the second sensor, determine a difference between the first voltage signal edge and the second voltage signal edge, and rotate transducer from the second position to the first position, the first position being a central position.

### CROSS-REFERENCE TO RELATED PATENT APPLICATIONS

This application claims the benefit of and priority to U.S. Provisional Patent Application No. 63/698,016, filed Sep. 23, 2024, which is incorporated herein by reference in its entirety and for all purposes.

### FIELD

Embodiments of the subject matter disclosed herein relate to ultrasound imaging, and more particularly, to an ultrasound probe having improved center position detection.

### BACKGROUND

During a medical imaging scan, a plurality of medical images of a patient are obtained by a technician, such as a sonographer, to measure or detect various aspects of anatomical features present within the medical images. In some instances, the medical images are obtained by inserting a probe (e.g., an endocavity probe) into an anatomical cavity (e.g., a vagina, a rectum, an esophagus) to obtain high-resolution images of nearby internal structures for diagnostic evaluation. Furthermore, external applications (e.g., off-system computing devices) may be used to process the medical images and provide various capabilities such as enhanced diagnostic tools.

### SUMMARY

One embodiment relates to an ultrasound imaging system including a probe. The probe includes a distal portion, a proximal portion, and a processing circuit. The distal portion includes a first magnet and a first sensor. The proximal portion is positioned at a head of the probe and includes a transducer, a second magnet, and a second sensor. The processing circuit includes a processor coupled to a memory device storing instructions thereon. The processing circuit is configured to detect a first voltage signal edge via the first magnet and the first sensor, rotate the transducer from a first position to a second position to locate a second voltage signal edge via the second magnet and the second sensor, determine a difference between the first voltage signal edge and the second voltage signal edge, and rotate transducer from the second position to the first position based on the difference being within a predetermined threshold, where the first position is a central position.

Another embodiment relates to an ultrasound probe including a distal portion, a proximal portion, and a processing circuit. The distal portion includes a first magnet and a first sensor. The proximal portion is positioned at a head of the probe and includes a transducer, a second magnet, and a second sensor. The processing circuit includes a processor coupled to a memory device storing instructions thereon. The processing circuit is configured to detect a first voltage signal edge via the first magnet and the first sensor, rotate the transducer from a first position to a second position to locate a second voltage signal edge via the second magnet and the second sensor, determine a difference between the first voltage signal edge and the second voltage signal edge, and rotate transducer from the second position to the first position based on the difference being within a predetermined threshold, where the first position is a central position.

Another embodiment relates to a system for initializing an ultrasound probe. The system includes a processing circuit with a processor coupled to a memory device storing instructions thereon. The processing circuit is configured to detect a first voltage signal edge via a first magnet and a first sensor positioned at a distal portion of the ultrasound probe. The processing circuit is configured to rotate, based on locating the first voltage signal edge, a transducer from a first position to a second position to locate a second voltage signal edge via a second magnet and a second sensor positioned at a proximal portion of the ultrasound probe. The processing circuit is configured to determine a difference between the first voltage signal edge and the second voltage signal edge. The processing circuit is configured to rotate the transducer from the second position to the first position based on the difference being within a predetermined threshold, where the first position is a central position.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is an illustration of an ultrasound imaging system, according to an example embodiment.

FIG. 2 is a block diagram of the ultrasound imaging system of FIG. 1, according to an example embodiment.

FIG. 3 is a block diagram of a processing circuit of the ultrasound imaging system of FIG. 2, according to an example embodiment.

FIG. 4 is a flow chart illustrating a method for performing an ultrasound imaging procedure, according to an example embodiment.

FIG. 5 is an illustration of a probe of the ultrasound imaging system of FIG. 1, according to an example embodiment.

FIG. 6A is a first illustration of internal components of the probe of FIG. 5, according to an example embodiment.

FIG. 6B is a second illustration of the internal components of the probe of FIG. 5, according to an example embodiment.

FIG. 6C is a third illustration of the internal components of the probe of FIG. 5, according to an example embodiment.

FIG. 7 is a flow chart illustrating a method for initializing the probe of FIG. 5, according to an example embodiment.

FIG. 8 is a flow chart illustrating a method for detecting an error during initialization of the probe of FIG. 5, according to an example embodiment.

### DETAILED DESCRIPTION

Referring generally to the figures, systems and methods for an ultrasound probe having improved center position detection are disclosed. More specifically, the systems and methods described herein include configuring components of the ultrasound probe within a tip and a handle of the ultrasound probe such that initialization of the ultrasound probe is improved and detection of faults within the system is enhanced.

In existing systems, a single hall-effect sensor of an ultrasound probe is located within a distal portion of the ultrasound probe (e.g., the handle). In such configurations, the single hall-effect sensor is mounted near a rotating axle of a motor within the handle, where the signal edge (e.g., falling edge, rising edge) indicates center position of the transducer with respect to an axis of the drive shaft during probe initialization. Furthermore, existing systems require more torque from the motor than a freely rotating motor axis as mechanical features positioned within the probe 106 are moved (e.g., adjusted, displaced) during initialization.

The systems and methods described herein provide a technical solution to existing systems by improving the reliability of the initialization process of an ultrasound probe. That is, by calculating the relative distance and/or angle between a motor hall-effect sensor positioned at the distal portion of the probe and a transducer hall-effect sensor at the proximal portion of the probe, the voltage signal edge present throughout the probe can be accurately represented, thereby improving voltage readings accuracy and enhancing reliability of the probe. Furthermore, capturing the voltage signal edge at both the distal portion and the proximal portion compared to existing ultrasound probes causes the ultrasound probe to output a more accurate initialization assessment, which decreases the likelihood of utilizing a defective probe during a medical procedure. As yet another technical solution, the systems and methods described herein provide an ability to store reference values in the system memory during manufacturing of the probe, resulting in consistency between initialization cycles.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

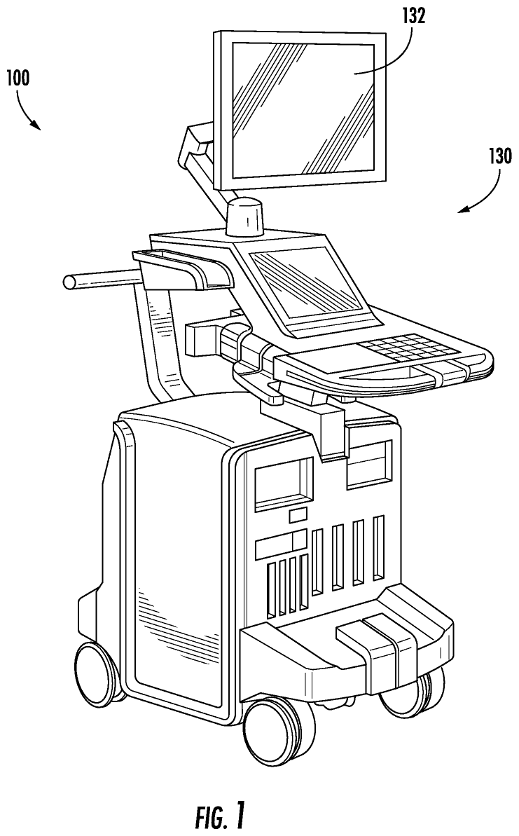

Referring to FIG. 1, an ultrasound imaging system 100 is shown. Specifically, the ultrasound imaging system 100 is shown to include a user interface 130 and a display device 132. The ultrasound imaging system 100 may be used in a medical environment (e.g., hospitals, clinics, etc.), As an example, by a sonographer, technician, or other clinician certified to collect ultrasound data from a patient. Although the systems and methods are described herein in the context of the ultrasound imaging system 100, it should be appreciated that the improved probe configuration described herein may be implemented using any of a variety of medical imaging systems (e.g., medical resonance imaging, x-ray, computed tomography, positron emission tomography, etc.).

The user interface 130 may be used by a sonographer or other clinician to control operation of the ultrasound imaging system 100. As an example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

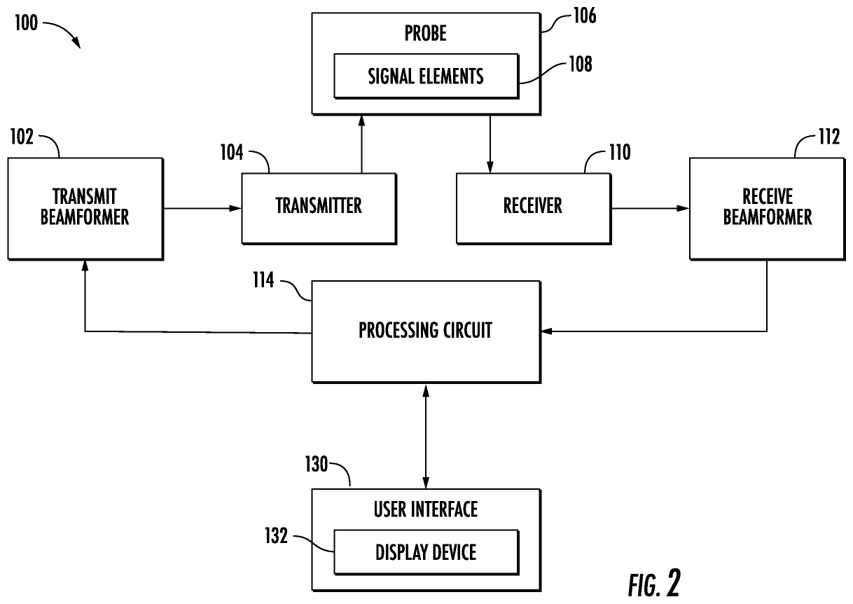

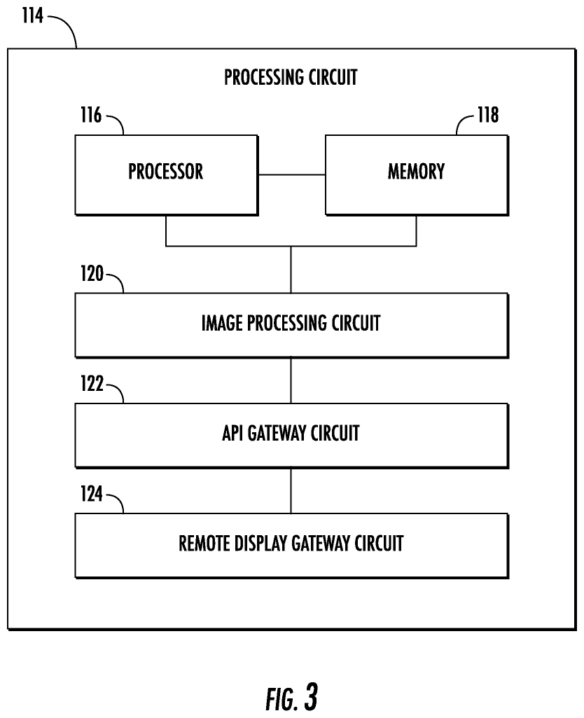

According to some embodiments, the user interface 130 may be physically separate from the rest of the ultrasound imaging system 100 (e.g., the transmit beamformer 102, the transmitter 104, the probe 106, the receiver 110, the receive beamformer 112, and/or the processing circuit 114, as shown in FIG. 2). The user interface 130 may communicate with a processor (e.g., processor 116 as shown in FIG. 3) through a wireless protocol, such as Wi-Fi, Bluetooth, wireless local area network (WLAN), near-field communication, and so on. According to some embodiments, the user interface 130 may communicate with the processor through an application programming interface (API). In some embodiments, the user interface 130 may include physical controls such as one or more of buttons, sliders, a rotary knob, a mouse, a keyboard, a trackball, hard keys linked to specific actions, soft keys that may be configured to control different functions, and so on.

As shown in FIG. 1, the ultrasound imaging system 100 may also include a display device 132. In some embodiments, as shown in FIG. 2, the display device 132 may be part of the user interface 130. The display device 132 may be a screen configured to display a graphical user interface (GUI) based on an instruction from a memory (e.g., memory 118, as shown in FIG. 3). The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. As an example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132.

The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring to FIG. 2, a block diagram of the ultrasound imaging system 100 is shown. As shown in FIG. 3, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. As an example, the anatomy being imaged may be identified as a fetal skull at a first location of the probe 106. Then, the position sensor may track the movement of the probe 106 relative to the fetal skull in order to identify successive locations of the probe 106. As another example, a cellular mass of interest (e.g., tumor) may be identified on a prostate during a biopsy procedure. Then, the position sensor may track the movement of the probe relative to the cellular mass of interest in order to identify successive locations of the probe. In some embodiments, the position sensor may transmit position data to be stored within the ultrasound imaging system 100 (e.g., in memory 118, as shown in FIG. 3).

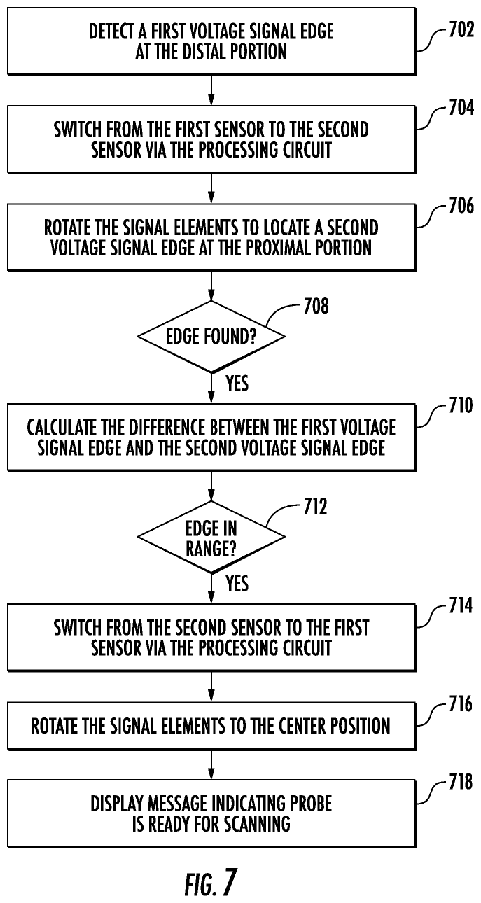

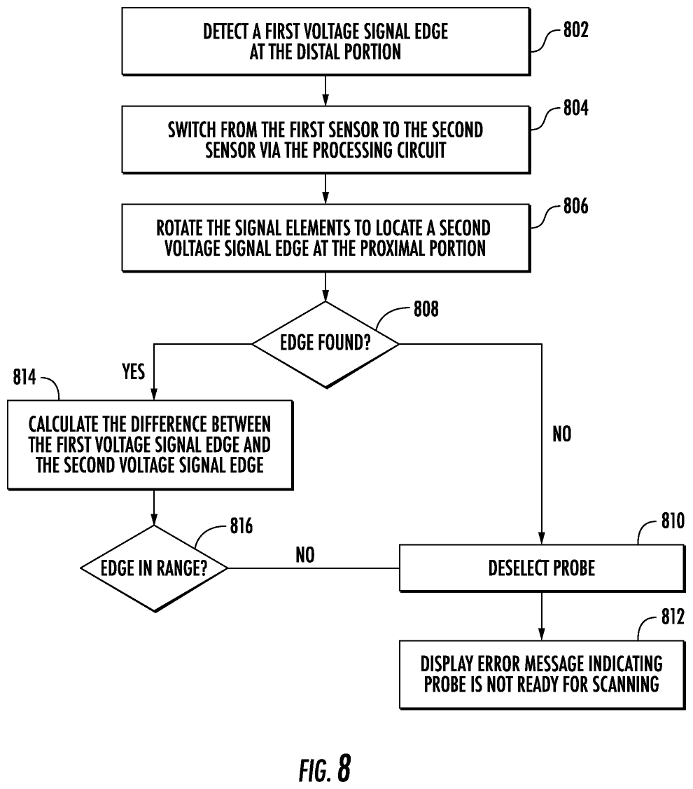

In some embodiments, the probe 106 may be an endocavity probe (e.g., an endocavitary transducer). An endocavity ultrasound probe refers to a probe configured to perform internal imaging procedures through body cavities (e.g., the vagina, the rectum, the esophagus, etc.). The endocavity probe may comprise a compact, elongated shape compared to an external (e.g., linear, curvilinear, etc.) probe. In this way, during an ultrasound imaging procedure, the endocavity probe may be configured to achieve proximity to internal organs (e.g., the uterus, ovaries, prostate, or rectal wall). By emitting high-frequency sound waves, the endocavity probe captures ultrasound images of such internal organs that may be used in diagnosing conditions such as pelvic abnormalities, early pregnancies, or prostate disorders. In some embodiments, endocavity probes may be used in gynecological, obstetric, fertility, urological, and transesophageal examinations offering enhanced image clarity compared to external ultrasound techniques (e.g., using a linear probe, a curvilinear probe, etc.). In some embodiments, endocavity probes may be used in biopsy procedures, in which the probe may guide a surgical tool (e.g., biopsy needle) during removal of a tumor in the cervix, rectum, esophagus, or various other anatomical cavities of a patient. In such embodiments, the probe 106 may include a biopsy needle guide removably coupled to the head of the probe 106. An example of an ultrasound imaging procedure using an endocavity probe is described in greater detail below, with reference to FIGS. 7 and 8.

The probe 106 may include a transducer configured to transmit and receive an ultrasound signal. In some embodiments, as shown in FIG. 2, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). As an example, during a fetal examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance match between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as shown in FIG. 3) may be configured to perform some or all of the functions associated with the receive beamformer 112.

Although the transmit beamformer 102, the transmitter 104, the receiver 110, and the receive beamformer 112 are shown in FIG. 2 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 2, the ultrasound imaging system 100 is shown to include a processing circuit 114. While shown as being separate from the probe 106 in FIG. 2, it will be appreciated that the processing circuit 114 can be part of the probe 106. As an example, the processing circuit 114 can be disposed in a handheld housing of the probe 106 (e.g., in the case of the probe 106 being a wireless probe).

The ultrasound imaging system 100 is also shown to include the user interface 130 and the display device 132, as described above in greater detail above with reference to FIG. 1.

Referring to FIG. 3, the processing circuit 114 of the ultrasound imaging system 100 is shown in greater detail. As shown, the processing circuit 114 may include at least one processor 116, a memory 118, an image processing circuit 120, an API gateway circuit 122, and a remote display gateway circuit 124. In this way, the processing circuit 114 may be structured or configured to execute or implement the instructions, commands, and/or control processes described herein with respect to the processor 116, the memory 118, the image processing circuit 120, the API gateway circuit 122, and the remote display gateway circuit 124.

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. As an example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. As an example, the operator may switch between modes using user interface 130 (e.g., using physical controls, interface inputs representing physical controls, etc.). While the term “image” or “images” are used herein to for the purposes of example, it will be appreciated that such terms cover still images as well as videos, clips, or a series of images for each. As an example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. As an example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/see, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. As an example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132.

The processing circuit 114 also includes the memory 118. The memory 118 may be configured to, as an example, store processed volumes of data obtained by the ultrasound imaging system 100 (e.g., ultrasound data collected by the probe 106, user inputs received by the user interface 130, etc.). As an example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. As an example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120. The image processing circuit 120 is configured to receive image data obtained by the transducer of the probe 106 during an ultrasound scan. The image data refers to ultrasound data collected by the probe 106 while performing an ultrasound examination on a patient. As an example, the image data may be collected during a fetal ultrasound and may therefore include various images of a patient's uterus and the fetal anatomy contained therein. The image processing circuit 120 may include multiple deep learning-based models configured to analyze the image data. As an example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

The processing circuit 114 may also include the API gateway circuit 122 and the remote display gateway circuit 124. Both the API gateway circuit 122 and the remote display gateway circuit 124 are configured to facilitate integration of the probe 106 with the ultrasound imaging system 100, as described herein. As an example, the API gateway circuit 122 may be configured to facilitate transmitting the ultrasound data from the probe 106 to the ultrasound imaging system 100, and the remote display gateway circuit 124 may be configured to facilitate transmitting display data (e.g., display data, as described below) from the ultrasound imaging system 100 to a remote display system.

The API gateway circuit 122 refers to an intermediary between the ultrasound imaging system 100 and the probe 106. The API gateway circuit 122 facilitates secure and efficient communication between the ultrasound imaging system 100 and the probe 106 by facilitating authentication, authorization, request routing, and data transformation. Furthermore, the API gateway circuit 122 provides the ultrasound imaging system 100 with access to additional functionality, such as image acquisition and processing, through standardized API calls. Using the API gateway circuit 122, the ultrasound imaging system 100 can support various external applications, including remote diagnostics, telemedicine, advanced data analysis, and so on.

The remote display gateway circuit 124 facilitates the transmission of ultrasound images and data (e.g., display data) to remote displays, enabling real-time monitoring and diagnostics from different locations. As an example, the remote display gateway circuit 124 may allow a user of the ultrasound imaging system 100 to interact with data being processed at the remote display system. In other words, the data may be viewed and interacted with at the ultrasound imaging system 100 while being processed at the remote display system in real-time. The remote display gateway circuit 124 communicates with remote devices (e.g., the ultrasound imaging system 100, the remote display system, etc.) via a network. As described herein, such communication allows users (e.g., sonographers, clinicians, etc.) to view and interact with ultrasound images (e.g., display data) on the display device 132.

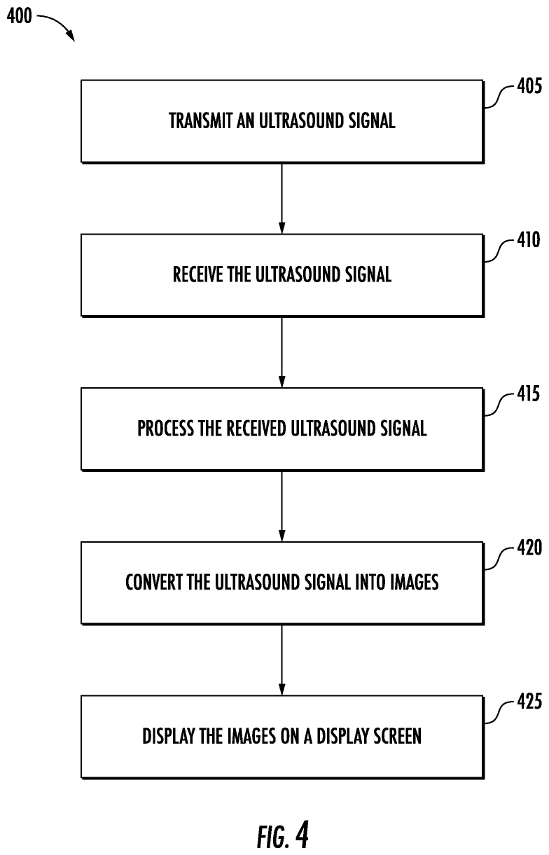

Referring to FIG. 4, a flow chart is shown illustrating a method 400 for performing an ultrasound imaging procedure using an ultrasound imaging system. In at least one embodiment, the ultrasound imaging system referred to by method 400 is the ultrasound imaging system 100 described above with reference to FIGS. 1-3, and method 400 may be implemented by the ultrasound imaging system 100. In some embodiments, method 400 may be implemented as executable instructions in a memory of the ultrasound imaging system 100, such as the memory 118 of FIG. 3.

At step 405, method 400 may include transmitting an ultrasound signal. The signal elements 108 (e.g., the transducer) of the probe 106 may transmit the ultrasound signal at step 405 by emitting high-frequency sound waves. For instance, when the transducer is brought into contact with a patient's body, the transducer sends the sound waves into the body. As the sound waves travel through different tissues and organs, they encounter boundaries between different types of tissues, such as muscle and bone. At these boundaries, some of the sound waves are reflected back to the transducer, while others continue to travel deeper into the body.

Then, at step 410, the transducer captures the sound waves that are reflected back from the tissue boundaries. These reflected waves, or echoes, are converted into electrical signals by the transducer. The strength and timing of these echoes provide information regarding the depth and density of the tissues they encountered at step 405 (which is used to create accurate images of the internal structures).

At step 415, the electrical signals are processed by ultrasound imaging system 100. In other words, once the ultrasound signals are received by the transducer and converted into electrical signals, the electrical signals undergo processing to enhance their quality and extract meaningful information. More specifically, the ultrasound imaging system 100 (e.g., the image processing circuit 120) may be configured to amplify the electrical signals and filter out noise. Advanced algorithms may be applied to analyze the echoes, determining the origin and intensity of the echoes. In some embodiments, step 415 may include adjusting parameters such as gain and depth to optimize the clarity of resulting images (e.g., displayed at step 425, as described below).

At step 420, the processed ultrasound signals are converted into ultrasound images. The ultrasound imaging system 100 is configured to translate the electrical signals into a visual format that represents the internal structures of the body. Specifically, step 420 may include mapping the intensity and location of the echoes to corresponding points on the ultrasound image.

At step 425, the ultrasound images are displayed on a display screen (e.g., display device 132) for real-time viewing. The ultrasound images may be displayed in grayscale, with varying shades representing different tissue densities. The ultrasound images can be adjusted for brightness, contrast, and other parameters to enhance visibility.

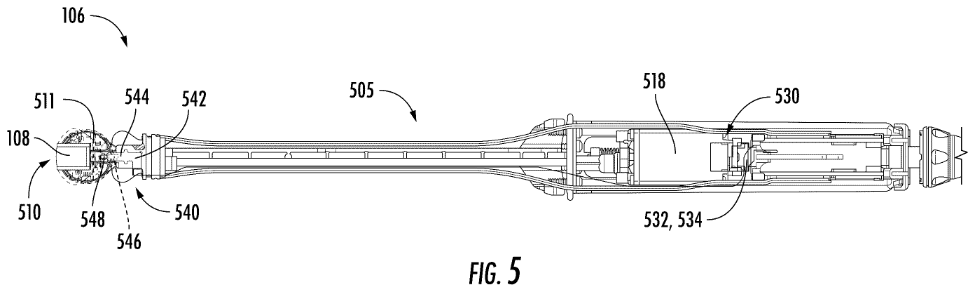

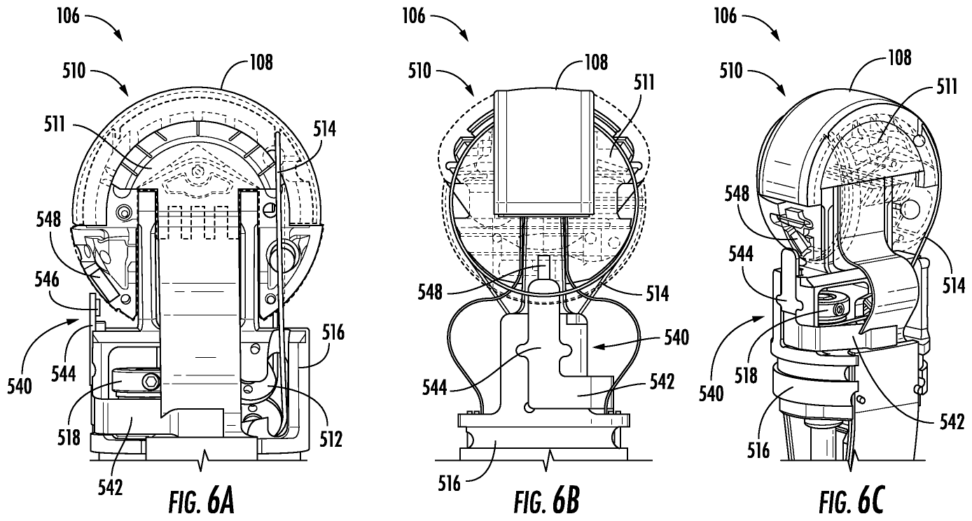

Referring to FIGS. 5-6C, the probe 106 is shown in greater detail. More specifically, the probe 106 of FIG. 5 may be an endocavity probe. The probe 106 includes a distal portion 505 and a proximal portion 510, such that the distal portion 505 extends away from the proximal portion 510. In some embodiments, the distal portion 505 is a handle of the probe 106. As an example, a sonographer may grip (e.g., hold, engage with) the distal portion 505 during an ultrasound imaging procedure (e.g., gynecological examination, obstetric examination, fertility examination, urological examination, transesophageal examination, etc.). In some embodiments, the proximal portion 510 is a tip of the probe 106. As an example, a sonographer may guide the proximal portion 510 in an anatomical cavity of a patient during an ultrasound imaging procedure.

The probe 106 is also shown to include the signal elements 108. As described above, the signal elements 108 may be arranged in a transducer array. The transmit beamformer 102 and the transmitter 104 drive the signal elements 108 to emit pulsed ultrasonic signals into a body of a subject (e.g., a patient). As an example, during a gynecological examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance match between a tissue to be imaged and a material of the transducer (e.g., such that the pulsed electronic signals can be back-scattered from the anatomical structures in the body and received as echoes by the signal elements 108), and a damping block configured to absorb ultrasound energy.

As shown in FIGS. 5-6C, the proximal portion 510 may include a transducer housing 511. The transducer housing 511 may be a protective plastic shell composed of a medical grade plastic. As an example, the transducer housing 511 may be composed of polyurethane (PU), polycarbonate (PC), acrylonitrile butadiene styrene (ABS), or various other medical grade plastics. The transducer housing 511 may be configured to enclose (e.g., house, protect) the internal components positioned at the proximal portion 510. As an example, the transducer housing 511 may enclose a rotation assembly and a sensor system at the head of the probe 106, as discussed in more detail below. An outermost surface of the transducer housing 511 may be configured to contact an anatomical cavity of the patient. In some embodiments, the transducer housing 511 may be substantially rounded in shape. In some embodiments, the transducer housing 511 may be substantially contoured in shape to accommodate various components within the head of the probe 106. In some embodiments, the transducer housing 511 may be configured to separate the various components within the head of the probe 106.

As shown in FIGS. 6A-6C, the proximal portion 510 may include a rotation assembly. The rotation assembly may be configured to rotate the transducer. The rotation system may include a pulley system 512 and at least one cord 514. The pulley system 512 may be configured to rotate the transducer (e.g., using a force imparted by a drive assembly 518). The pulley system 512 may include at least one cord 514 (e.g., a string, rope, etc.) configured to intersect (e.g., cross) at an intersection point in the proximal portion 510. Due to the intersection point, the pulley system 512 (e.g., using a force provided by the drive assembly 518) is configured to pull the at least one cord 514 at an angle defined by the intersection point. In turn, the transducer may be rotated and angled up to a sweep angle of 180 degrees from side-to-side (e.g., left-to-right). In some cases, the transducer can sweep to 170 degrees from side-to-side (e.g., left-to-right). In some cases, the transducer can sweep up to 160 degrees from side-to-side (e.g., left-to-right) using the pulley system 512. It will be appreciated that the pulley system 512 can include any number of pulleys to guide and control the at least one cord 514 to ensure the cord 514 does not interfere with other components of the probe 106 (e.g., ensure the at least one cord 514 does not interface with an interior wall of the probe 106 or cause friction forces during operation). As an example, the pulley system 512 could include, one pulley, two pulleys, three pulleys, four pulleys, five pulleys, six pulleys or more, with any number of the pulleys positioned in the distal portion 505 of the probe 106 or in the proximal portion 510 of the probe 106. As an example, in some embodiments, two or more pulleys 512 are located in at least one of the distal portion 505 or the proximal portion 510.

The probe 106 may include a stationary carrier 516. The stationary carrier 516 may refer to a mechanical component (e.g., rigid material, contoured component, block) disposed between the distal portion 505 and the proximal portion 510, thereby acting as a mechanical bridge between the distal portion 505 and the proximal portion 510. The stationary carrier 516 may be configured to provide structural support to the internal components of the head of the probe 106. As an example, the stationary carrier 516 may provide a rigid mounting base for the signal elements 108.

As shown in FIGS. 5-6C, the probe 106 may include a drive assembly 518. The drive assembly 518 may include a drive shaft, a wheel, and a motor. The drive shaft may be configured to transmit a force (e.g., rotational or linear motion) from the motor to the transducer (e.g., the signal elements 108). In turn, the force imparted on the transducer may cause the probe 106 to sweep an ultrasound beam across a region of interest (e.g., uterus, ovaries, prostate, rectal wall, esophagus, etc.), thereby producing ultrasound images of the region of interest. The wheel may be configured to transmit a force (e.g., rotational or linear motion) from the motor to the sensing components of the distal portion 505 (e.g., the handle sensor system 530). In some embodiments, at least a portion of the drive assembly 518 (e.g., a portion of the drive shaft, a portion of the wheel, the motor, etc.) may be disposed within the distal portion 505 of the probe 106. As an example, the drive assembly 518 may be positioned within an interior portion of the handle. In some embodiments, the drive assembly 518 may include a first end configured to drive various internal components of the distal portion 505 via the wheel and a second end configured to drive various internal components of the proximal portion 510 via the drive shaft.

The probe 106 may include a sensor system. The sensor system may be configured to monitor the probe 106 during operation (e.g., a medical imaging procedure). The sensor system may include a handle sensor system 530 and a probe head sensor system 540. In some embodiments, the first end of the drive assembly 518 may be configured to drive the rotation assembly and the probe head sensor system 540 during initialization of the probe 106. In some embodiments, the second end of the drive assembly 518 may be configured to drive the handle sensor system 530 during initialization of the probe 106.

Referring to FIG. 5, the sensor system may include a handle sensor system 530. The handle sensor system 530 may be positioned at the distal portion 505. The handle sensor system 530 may include a first sensor 532 and a first magnet 534. The handle sensor system 530 may be configured to initialize the probe 106 prior to performing a medical imaging procedure. The handle sensor system 530 may be configured to communicate with the probe head sensor system 540 during initialization of the probe 106.

The handle sensor system 530 may include first sensor 532 (e.g., motor sensor, motor hall sensor). The first sensor 532 may include a hall-effect sensor such that the first sensor 532 is configured to detect the presence, strength, and direction of a magnetic field within the distal portion 505. The first sensor 532 may be positioned at an interior of the handle. In some embodiments, the first sensor 532 may be positioned on a flexible printed circuit board (PCB) positioned within the interior of the handle. In some embodiments, the first sensor 532 may be positioned proximate to the second end of the drive assembly 518 within the interior of the handle.

The handle sensor system 530 may include first magnet 534. The first magnet 534 may be positioned proximate to the first sensor 532 at the distal portion 505. As an example, the first magnet 534 may be positioned in an interior of the handle, such that a portion of the interior of the handle encloses the first magnet 534, thereby creating a barrier between (e.g., physically separating) the first sensor 532 and the first magnet 534 within the interior of the handle. In some embodiments, the first magnet may be positioned in a magnet carrier within the interior of the handle. In some embodiments, the pole of the first magnet 534 may be positioned directly at a face of the first sensor 532. In some embodiments, the first sensor 532 and the first magnet 534 are positioned such that a falling edge is formed (e.g., a change from a high level of voltage to a low level of voltage). As an example, when the first magnet 534 is aligned with the first sensor 532, the first sensor 532 provides a signal indicating the voltage level has adjusted from a high voltage level to a low voltage level. The voltage change may include dropping from a high voltage level of a range between about 3-5V to a low voltage level of about 0V. In some embodiments, the first sensor 532 and the first magnet 534 are positioned such that a rising edge is formed (e.g., a change from a low level of voltage to a high level of voltage). As an example, when the first magnet 534 is aligned with the first sensor 532, the first sensor 532 provides a signal indicating the voltage level has adjusted from a low voltage level to a high voltage level. In some embodiments, the voltage change may include rising from a low voltage level of about 0V to a high voltage level of a range between about 3-5V.

Referring to FIGS. 6A-6C, the sensor system may include a probe head sensor system 540. The probe head sensor system 540 may be positioned at the proximal portion 510. The probe head sensor system 540 may include a flexible PCB 542, a stiffener 544, a second sensor 546, and a second magnet 548. The probe head sensor system 540 may be configured to initialize the probe 106 prior to performing a medical imaging procedure.

The probe head sensor system 540 may include a flexible printed circuit board (PCB) 542. The flexible PCB 542 may be mounted to the stationary carrier 516. As an example, the flexible PCB 542 may abut (e.g., positioned adjacent to, positioned directly on) at least a portion of the stationary carrier 516. As another example, the flexible PCB 542 may wrap (e.g., bend around) at least a portion of the stationary carrier 516. The flexible PCB 542 may be configured to electrically support various components of the probe 106. The flexible PCB 542 may be configured to route signals from the proximal portion 510. As an example, the flexible PCB 542 may be configured to route electrical signals from the signal elements 108 to the electrical components positioned at the distal portion 505.

The flexible PCB 542 may include a stiffener 544. The stiffener 544 may include a layer of rigid material bonded to the flexible PCB 542. As an example, the stiffener 544 may include fiberglass epoxy laminate (FR4), aluminum, polyether ketone (PEEK), titanium, or various other medical-grade rigid materials. In some embodiments, the stiffener 544 may be positioned at an outer face of the flexible PCB 542. In some embodiments, the stiffener 544 may extend from an upper portion of the flexible PCB 542, thereby creating a rigid extension portion. The stiffener 544 may be configured to receive one or more sensors of the probe head sensor system 540 to prevent undesired bending and ensure the one or more sensors are held in place during operation of the probe 106.

The probe head sensor system 540 may include a second sensor 546 (e.g., transducer sensor, transducer hall sensor). The second sensor 546 may include a hall-effect sensor, such that the second sensor 546 is configured to detect the presence, strength, and direction of a magnetic field within the proximal portion 510. The second sensor 546 may be positioned on the flexible PCB 542. As an example, the second sensor 546 may be positioned on an interior surface of the stiffener 544, such that the second sensor 546 faces the internal components at the proximal portion 510. As another example, the second sensor 546 may be positioned on an outer face of the flexible PCB 542.

The probe head sensor system 540 may include second magnet 548. The second magnet 548 may be positioned proximate to the second sensor 546 at the proximal portion 510. As an example, the second magnet 548 may be positioned in the transducer housing 511, such that transducer housing 511 encloses the second magnet 548, thereby creating a barrier between (e.g., physically separating) the second sensor 546 and the second magnet 548. In some embodiments, the pole of the second magnet 548 may be positioned directly at a face of the second sensor 546. In some embodiments, the second sensor 546 and the second magnet 548 are positioned such that a falling edge is formed (e.g., a change from a high level of voltage to a low level of voltage). As an example, when the second magnet 548 is aligned with the second sensor 546, the second sensor 546 provides a signal indicating the voltage level has adjusted from a high voltage level to a low voltage level. The voltage change may include dropping from a high voltage level of a range between about 3-5V to a low voltage level of about 0V. In some embodiments, the pole of the second magnet 548 may be positioned directly at a face of the second sensor 546. In some embodiments, the second sensor 546 and the second magnet 548 are positioned such that a rising edge is formed (e.g., a change from a low level of voltage to a high level of voltage). As an example, when the second magnet 548 is aligned with the second sensor 546, the second sensor 546 provides a signal indicating the voltage level has adjusted from a low voltage level to a high voltage level. The voltage change may include rising from a low voltage level of about 0V to a high voltage level of a range between about 3-5V.

It should be understood that the sensor system can include any number of sensors. As an example, the sensor system may include a first hall sensor at the distal portion 505 and a second hall sensor in the proximal portion 510. In some embodiments, the sensor system may include more than one hall sensor positioned at least one of the distal portion 505 or the proximal portion 510. As an example, the sensor system may include two hall sensors positioned at the distal portion 505 and one hall sensor positioned at the proximal portion 510. As another example, the sensor system may include one hall sensors positioned at the distal portion 505 and two hall sensor at the proximal portion 510. As yet another example, the sensor system may include two hall sensors positioned at the distal portion 505 and two hall sensor positioned at the proximal portion 510. As yet another example, the sensor system may include more than two hall sensors positioned at the distal portion 505 and one hall sensor positioned at the proximal portion 510. As yet another example, the sensor system may include one hall sensors positioned at the distal portion 505 and more than two hall sensor positioned at the proximal portion 510. As yet another example, the sensor system may include more than two hall sensors positioned at the distal portion 505 and more than two hall sensors positioned at the proximal portion 510.

It should be understood that the sensor system can include any number of magnets. As an example, the sensor system may include a first magnet at the distal portion 505 and a second magnet in the proximal portion 510. In some embodiments, the sensor system may include more than one magnet positioned at least one of the distal portion 505 or the proximal portion 510. As an example, the sensor system may include two magnets positioned at the distal portion 505 and one magnet positioned at the proximal portion 510. As another example, the sensor system may include one magnet positioned at the distal portion 505 and two magnets at the proximal portion 510. As yet another example, the sensor system may include two magnets positioned at the distal portion 505 and two magnets positioned at the proximal portion 510. As yet another example, the sensor system may include one magnet positioned at the distal portion 505 and more than two magnets positioned at the proximal portion 510. As yet another example, the sensor system may include more than two magnets positioned at the distal portion 505 and one magnet positioned at the proximal portion 510. As yet another example, the sensor system may include more than two magnets positioned at the distal portion 505 and more than two magnets positioned at the proximal portion 510.

Referring now to FIG. 7, a method 700 for initializing an ultrasound probe prior to an ultrasound imaging procedure is shown. In at least one embodiment, the ultrasound probe referred to by method 700 is the probe 106 described above with reference to FIGS. 5-6C, and method 700 may be implemented by the ultrasound imaging system 100. In some embodiments, method 700 may be implemented as executable instructions in a memory of the ultrasound imaging system 100, such as the memory 118 of FIG. 3. According to various example instances, the ultrasound imaging procedure referred to by method 700 may be an ultrasound imaging procedure (e.g., gynecological examination, obstetric examination, fertility examination, urological examination, transesophageal examination) and/or a biopsy procedure.

As shown, at step 702, the processing circuit 114 may detect a first signal from the first sensor positioned at the distal portion 505 of the probe 106. The signal may be a first voltage signal edge (e.g., first falling edge, first rising edge, motor hall edge). The drive assembly 518 may generate a force (e.g., torque), which is transmitted through the drive assembly 518 (e.g., via the wheel positioned at the first end) to the distal portion 505. As the first magnet 534 rotate about the distal portion 505 via the drive assembly 518, the first magnet 534 may move relative to the first sensor 532. Once the first magnet 534 is positioned within a distance threshold (e.g., release threshold) of the first sensor 532, the first sensor 532 may provide a signal indicating the presence of the first voltage signal edge. In some embodiments, the voltage level may transition from a high voltage level to a low voltage level of a range between about 3-5V to a low voltage level of about 0V, creating a falling edge. In some embodiments, the voltage level may transition from a low voltage level of about 0V to a high voltage level of a range between about 3-5V, creating a rising edge. The first voltage signal edge may indicate that the signal elements 108 are in a center position, such that the signal elements 108 are positioned in a midpoint (e.g., neutral position) of travel within its scanning range.

At step 704, the processing circuit 114 may switch (e.g., adjust, change) from the first sensor 532 to the second sensor 546 positioned at the proximal portion 510 of the probe. The processing circuit 114 may switch to the second sensor 546 to adjust the position reference source at which the probe 106 relies upon as when synchronizing data during the initialization process. When the processing circuit 114 switches to the second sensor 546, the processing circuit 114 may begin logging data detected via the second sensor 546 rather than the first sensor 532.

At step 706, the processing circuit 114 may rotate the signal elements 108 to locate a second voltage signal edge (e.g., second falling edge, transducer hall edge) via the rotation assembly and the drive assembly 518. The signal elements 108 may rotate from a first position (e.g., center position) to a second position. The drive assembly 518 may generate a force (e.g., torque), which is transmitted through the drive assembly 518 (e.g., via the drive shaft) to the proximal portion 510, thereby engaging the rotation assembly (e.g., pulley system 512 and the at least one cord 514). As the signal elements 108 rotate at the proximal portion 510 via the rotation assembly and the drive assembly 518, the second magnet 548 moves relative to the second sensor 546.

At step 708, the processing circuit 114 may determine if the second voltage signal edge is present at the proximal portion 510. Once the second magnet 548 is positioned within a distance threshold (e.g., release threshold) of the second sensor 546, the second sensor 546 may provide a signal indicating the presence of the second voltage signal edge. In some embodiments, the voltage level may transition from a high voltage level to a low voltage level of a range between about 3-5V to a low voltage level of about 0V, creating a falling edge. In some embodiments, the voltage level may transition from a low voltage level of about 0V to a high voltage level of a range between about 3-5V, creating a rising edge.

At step 710, the processing circuit 114 may calculate a difference between the first voltage signal edge and the second voltage signal edge. The processing circuit 114 may then compare the calculated difference to a threshold range to determine if the first voltage signal edge and the second voltage signal edge are acceptable for operation of the probe 106. As an example, the calculated difference may indicate that the first sensor 532 and the second sensor 546 are aligned within allowable tolerances dictated by the threshold range.

At step 712, the processing circuit 114 may determine if the calculated difference between the first voltage signal edge and the second voltage signal edge falls within a threshold range (e.g., predetermined voltage signal edge, predetermined signal edge). The processing circuit 114 my compare the calculated difference to the threshold range set within the probe 106. In some embodiments, the threshold range may be programmed in the probe 106 during manufacture. In some embodiments, the threshold range may be adjusted by a user (e.g., sonographer, device technician) based on one or more inputs such as age of the probe, usage history, environmental factors (e.g., temperature, humidity), and various other inputs. In some embodiments, the threshold range may be defined in terms of arc degree values. As an example, the threshold range may include a range less than ±5.0° (e.g., ±1.0°, ±2.0°, ±3.0°, ±4.0°). As another example, the threshold range may include a range of about ±5.0°. In some embodiments, the arc degree values may be related to angle offset from center position.

At step 714, the processing circuit 114 may switch (e.g., adjust, change) from the second sensor 546 to the first sensor 532 positioned at the proximal portion 510 of the probe. The processing circuit 114 may switch to the first sensor 532 to adjust the position reference source at which the probe 106 relies upon as when synchronizing data during the initialization process.

At step 716, the processing circuit 114 may rotate the signal elements 108 from the second position to the first position via the rotation assembly and the drive assembly 518. The drive assembly 518 may generate a force (e.g., torque), which is transmitted through the drive assembly 518 (e.g., via the drive shaft) to the proximal portion 510, thereby engaging the rotation assembly (e.g., pulley system 512 and the at least one cord 514). As the signal elements 108 rotate at the proximal portion 510 via the rotation assembly and the drive assembly 518, the probe returns to the center position, thereby completing the initialization process.

At step 718, the processing circuit 114 may display a message, indicating initialization is complete. The processing circuit 114 may be communicatively coupled to the user interface 130. The processing circuit 114 may deliver a signal containing a message stating the probe 106 is ready for an ultrasound imaging procedure to the user interface 130. The user interface 130 may display the message on the ultrasound imaging system 100 for interpretation by a sonographer, technician, or other clinician certified to collect ultrasound data from a patient. In some embodiments, the processing circuit 114 may display the message on a remote display device (e.g., an off-the-shelf consumer electronic device such as a smartphone, a tablet, a laptop).

Referring now to FIG. 8, a method 800 for detecting an error when initializing an ultrasound probe prior to an ultrasound imaging procedure is shown. In at least one embodiment, the ultrasound probe referred to by method 800 is the probe 106 described above with reference to FIGS. 5-6C, and method 800 may be implemented by the ultrasound imaging system 100. In some embodiments, method 800 may be implemented as executable instructions in a memory of the ultrasound imaging system 100, such as the memory 118 of FIG. 3. According to various example instances, the ultrasound imaging procedure referred to by method 800 may be a gynecological examination, an obstetric examination, or a urological examination.

At step 802, the processing circuit 114 may detect a signal from the first sensor positioned at the distal portion 505 of the probe 106. The signal may be a first voltage signal edge (e.g., first falling edge, motor hall edge). The drive assembly 518 may generate a force (e.g., torque), which is transmitted through the drive assembly 518 (e.g., via the drive shaft) to the distal portion 505. As the first magnet 534 rotate about the distal portion 505 via the drive assembly 518, the first magnet 534 moves relative to the first sensor 532. Once the first magnet 534 is positioned within a distance threshold (e.g., release threshold) of the first sensor 532, the first sensor 532 may provide a signal indicating the presence of the first voltage signal edge. In some embodiments, the voltage level may transition from a high voltage level to a low voltage level of a range between about 3-5V to a low voltage level of about 0V, creating a falling edge. In some embodiments, the voltage level may transition from a low voltage level of about 0V to a high voltage level of a range between about 3-5V, creating a rising edge. The first voltage signal edge may indicate that the signal elements 108 are in a center position, such that the signal elements 108 are positioned in a midpoint (e.g., neutral position) of travel within its scanning range.

At step 804, the processing circuit 114 may switch (e.g., adjust, change) the designated sensor in which a position reference source is taken. The processing circuit 114 may switch from the first sensor to the second sensor positioned at the proximal portion 510 of the probe.

At step 806, the processing circuit 114 may rotate the signal elements 108 to locate a second voltage signal edge (e.g., second falling edge, transducer hall edge) via the rotation assembly and the drive assembly 518. The signal elements 108 may rotate from a first position (e.g., center position) to a second position. The drive assembly 518 may generate a force (e.g., torque), which is transmitted through the drive assembly 518 (e.g., via the drive shaft) to the proximal portion 510, thereby engaging the rotation assembly (e.g., pulley system 512 and the at least one cord 514). As the signal elements 108 rotate at the proximal portion 510 via the rotation assembly and the drive assembly 518, the second magnet 548 moves relative to the second sensor 546. At step 808, the processing circuit 114 may determine if the second voltage signal edge is present at the proximal portion 510. Once the second magnet 548 is positioned within a distance threshold (e.g., release threshold) of the second sensor 546, the second sensor 546 may provide a signal indicating the presence of the second voltage signal edge. In some embodiments, the voltage level may transition from a high voltage level to a low voltage level of a range between about 3-5V to a low voltage level of about 0V, creating a falling edge. In some embodiments, the voltage level may transition from a low voltage level of about 0V to a high voltage level of a range between about 3-5V, creating a rising edge.

If the processing circuit 114 fails to locate a second voltage signal edge, the probe 106 may enter step 810 and step 812. At step 810, the processing circuit 114 may deselect the probe 106. In doing so, the processing circuit 114 may prevent signal communication between the ultrasound imaging system 100 and the probe 106. At step 812, the processing circuit 114 may display a message, indicating initialization is incomplete. The processing circuit 114 may be communicatively coupled to the user interface 130. The processing circuit 114 may deliver a signal containing a message stating the probe 106 not accepted for scanning to the user interface 130. The user interface 130 may display the message on the ultrasound imaging system 100 for interpretation by a sonographer, technician, or other clinician certified to collect ultrasound data from a patient. In some embodiments, the processing circuit 114 may display the message on a remote display (e.g., an off-the-shelf consumer electronic device such as a smartphone, a tablet, a laptop). In some embodiments, the message may include additional details regarding troubleshooting the error. In some embodiments, the message may include symbols estimating where the error has been located (e.g., a torn, derailed, or otherwise defective cord 514, loose components with the distal portion 505 or proximal portion 510).

At step 814, if the second voltage signal edge is found, the processing circuit 114 may calculate a difference between the first voltage signal edge and the second voltage signal edge and determine if the difference falls within a threshold range (e.g., predetermined signal edge, predetermined falling edge, predetermined rising edge). The processing circuit 114 may then compare the calculated difference to a threshold range to determine if the first voltage signal edge and the second voltage signal edge are acceptable for operation of the probe 106. As an example, the calculated difference may indicate that the first sensor 532 and the second sensor 546 are aligned within allowable tolerances dictated by the threshold range. As another example, the calculated difference may indicate that the first sensor 532 and the second sensor 546 are not aligned within allowable tolerances dictated by the threshold range, indicating an error in the probe 106.

At step 816, the processing circuit 114 may determine if the calculated difference between the first voltage signal edge and the second voltage signal edge falls within a threshold range. The processing circuit 114 my compare the calculated difference to the threshold range set within the probe 106. In some embodiments, the threshold range may be programmed in the probe 106 during manufacture. In some embodiments, the threshold range may be adjusted by a user (e.g., sonographer, device technician) based on one or more inputs such as age of the probe 106, usage history, environmental factors (e.g., temperature, humidity), and various other inputs. In some embodiments, the threshold range may be defined in terms of arc degree values. As an example, the threshold range may include a range less than ±5.0° (e.g., ±1.0°, ±2.0°, ±3.0°, ±4.0°). As another example, the threshold range may include a range of about ±5.0°. In some embodiments, the arc degree values may be related to angle offset from center position.

If the difference between the first voltage signal edge and the second voltage signal edge falls outside the threshold range, the probe 106 may enter step 810 and step 812. As discussed above, at step 810, the processing circuit 114 may deselect the probe 106, and, at step 812, the processing circuit 114 may display a message on the user interface 130, indicating initialization is incomplete.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112 (f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. As an example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, As an example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. As an example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, As an example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a probe comprising:
a distal portion comprising a first magnet and a first sensor; and
a proximal portion comprising a transducer, a second magnet, and a second sensor, the proximal portion positioned at a head of the probe;
a processing circuit comprising a processor coupled to a memory device storing instructions thereon, the processing circuit configured to:
detect a first voltage signal edge via the first magnet and the first sensor;
rotate, based on locating the first voltage signal edge, the transducer from a first position to a second position to locate a second voltage signal edge via the second magnet and the second sensor;
determine a difference between the first voltage signal edge and the second voltage signal edge; and
rotate, based on the difference being within a predetermined threshold, the transducer from the second position to the first position, the first position being a central position.

2. The ultrasound imaging system of claim 1, wherein the processing circuit is further configured to display a message on a user interface communicatively coupled to the probe during initialization of the probe.

3. The ultrasound imaging system of claim 2, wherein the processing circuit is further configured to:
based on failing to locate the second voltage signal edge, deselect the probe and display an error message on the user interface.

4. The ultrasound imaging system of claim 2, wherein the processing circuit is further configured to:
based on the difference being outside of the predetermined threshold, deselecting the probe and displaying an error message on the user interface.

5. The ultrasound imaging system of claim 1, further comprising a motor, the motor including:
a first end configured to facilitate movement of the first magnet,
a second end configured to drive a rotation assembly in the head, the rotation assembly configured to facilitate movement of the transducer and the second magnet.

6. The ultrasound imaging system of claim 1, wherein the probe is an endocavity probe, and the distal portion is a handle of the endocavity probe.

7. The ultrasound imaging system of claim 1, wherein at least one of the first sensor or the second sensor are hall-effect sensors.

8. The ultrasound imaging system of claim 1, wherein the first sensor is positioned on a flexible printed circuit board (PCB), the flexible PCB mounted to a stationary carrier of the probe.

9. The ultrasound imaging system of claim 1, further comprising a transducer housing, the transducer housing enclosing the second magnet at the head, thereby creating a barrier between the second sensor and the second magnet.

10. An ultrasound probe comprising:
a distal portion comprising a first magnet and a first sensor; and
a proximal portion comprising a transducer, a second magnet, and a second sensor, the proximal portion positioned at a head of the ultrasound probe; and
a processing circuit comprising a processor coupled to a memory device storing instructions thereon, the processing circuit configured to:
detect a first voltage signal edge via the first magnet and the first sensor;
rotate, based on locating the first voltage signal edge, the transducer from a first position to a second position to locate a second voltage signal edge via the second magnet and the second sensor;
determine a difference between the first voltage signal edge and the second voltage signal edge; and
rotate, based on the difference being within a predetermined threshold, the transducer from the second position to the first position, the first position being a central position.

11. The ultrasound probe of claim 10, wherein the processing circuit is further configured to display a message on a user interface communicatively coupled to the ultrasound probe during initialization of the ultrasound probe.

12. The ultrasound probe of claim 11, wherein the processing circuit is further configured to:
based on failing to locate the first voltage signal edge, deselect the ultrasound probe and display an error message on the user interface.

13. The ultrasound probe of claim 11, wherein the processing circuit is further configured to:
based on the difference being outside of the predetermined threshold, deselecting the ultrasound probe and displaying an error message on the user interface.

14. The ultrasound probe of claim 11, further comprising a motor, the motor including:
a first end configured to facilitate movement of the first magnet,
a second end configured to drive a rotation assembly in the head, the rotation assembly configured to facilitate movement of the transducer and the second magnet.

15. The ultrasound probe of claim 10, wherein the ultrasound probe is an endocavity probe, and the distal portion is a handle of the endocavity probe.

16. The ultrasound probe of claim 10, wherein at least one of the first sensor or the second sensor are hall-effect sensors.

17. The ultrasound probe of claim 10, wherein the first sensor is positioned on a flexible printed circuit board (PCB), the flexible PCB mounted to a stationary carrier of the ultrasound probe.

18. The ultrasound probe of claim 10, further comprising a transducer housing, the transducer housing enclosing the second magnet at the head, thereby creating a barrier between the second sensor and the second magnet.

19. A system for initializing an ultrasound probe comprising:
a processing circuit comprising a processor coupled to a memory device storing instructions thereon, the processing circuit configured to:
detect a first voltage signal edge via a first magnet and a first sensor positioned at a distal portion of the ultrasound probe;
rotate, based on locating the first voltage signal edge, a transducer from a first position to a second position to locate a second voltage signal edge via a second magnet and a second sensor positioned at a proximal portion of the ultrasound probe;
determine a difference between the first voltage signal edge and the second voltage signal edge; and
rotate, based on the difference being within a predetermined threshold, the transducer from the second position to the first position, the first position being a central position.

20. The system of claim 19, wherein the processing circuit is further configured to display a message on a user interface communicatively coupled to the ultrasound probe during initialization of the ultrasound probe.
